(19) United States
(12) Patent Application Publication
(10) Pub. No.: US 2026/0295127 A1
(43) Pub. Date: Oct. 1, 2026

(54) MEDICAL FILM AND PRODUCTION METHOD THEREFOR

(71) Applicant: KUREHA CORPORATION, Tokyo (JP)

(72) Inventors: Hironao SHIMADA, Tokyo (JP); Takashi MASAKI, Tokyo (JP); Fujio SEKINE, Tokyo (JP); Shin SUZUKI, Tokyo (JP)

(73) Assignee: KUREHA CORPORATION, Tokyo (JP)

(21) Appl. No.: 19/674,499

(22) Filed: May 12, 2026

Related U.S. Application Data
(63) Continuation-in-part of application No. 19/111,633, filed on May 9, 2024.

(30) Foreign Application Priority Data
May 9, 2023 (JP) 2023-077268

(51) Int. Cl. A61L 31/12; A61L 31/14

(52) U.S. Cl. CPC A61L 31/129; A61L 31/148

(57) ABSTRACT

A medical film containing a bioabsorbable polymer compound and PGA dispersoids or aggregates of PGA particles. A number-average molecular weight of the polymer is 7,500 or more and 100,000 or less, an average particle size of the particles is 1 μm or more and 60 μm or less, the content of the PGA dispersoids per 100 parts by weight of the bioabsorbable polymer compound is 2 parts by weight or more and 40 parts by weight or less, and a coefficient of variation CV of the number of particles contained in rectangular regions is 0.6 or less, where the coefficient of variation CV is determined from the number of particles measured in each of 16 rectangular regions with a size of 5 mm×4 mm set so as not to overlap with each other on a surface of the medical film.

### CROSS-REFERENCE TO RELATED APPLICATIONS

This application is a continuation-in-part of U.S. patent application Ser. No. 19/111,633, filed on Mar. 13, 2025, which is a national stage application under 35 U.S.C. § 371 of International Application No. PCT/JP2024/017274, filed on May 9, 2024, which claims priority to Japanese Patent Application No. 2023-077268, filed on May 9, 2023. The entire disclosures of the foregoing applications are incorporated herein by reference.

### TECHNICAL FIELD

The present invention relates to a medical film and a method for producing the medical film.

### BACKGROUND ART

Medical films (adhesion barrier materials) for preventing adhesion between organs are known. Such a film is attached to an organ after surgery to prevent adhesion between the organ and another organ when a wound is healing. Hyaluronic acid or the like is used for the adhesion barrier material (U.S. Pat. No. 5,017,229). However, since hyaluronic acid has a high dissolution rate in water, a molded article such as a film containing hyaluronic acid has a problem that it is difficult to retain the shape of the molded article for a long period in a living body or on a body surface where moisture such as body fluid is also present.

As a method for reducing the water solubility of hyaluronic acid, a method using a crosslinking reaction that utilizes a carboxyl group present in a molecule of hyaluronic acid is known. U.S. Pat. No. 6,294,202 discloses an adhesion barrier film using a composition containing a crosslinked polymer material (bioabsorbable polymer compound) rendered water-insoluble by crosslinking hyaluronic acid, carboxymethyl cellulose, carboxymethyl amylose, or the like. U.S. Pat. No. 6,294,202 further describes an adhesion barrier film in which one or both surfaces of a crosslinked polymer material are coated with a hydrophobic bioabsorbable polymer, such as polyglycolide, (D-, L-, DL-) polylactide, polydioxane, or a copolymer thereof, for the purpose of improving mechanical strength or extending the residence time in a living body. However, the adhesion barrier film of U.S. Pat. No. 6,294,202 has a problem that the shape thereof cannot be retained in a living body for a long period. In addition, fibers of a hydrophobic bioabsorbable polymer such as polyglycolide have high rigidity and thus reduce the flexibility of the adhesion barrier film.

A medical film containing a crosslinked polymer material and a particulate disintegration retardant containing a biodegradable polyester, such as polyglycolic acid (PGA), can retain its shape in a living body for a long period (WO 2021/201150). WO 2021/201150 describes that the disintegration retardant releases an acid to suppress the disintegration of the shape of the medical film. In addition, WO 2021/201150 describes that PGA with a lower molecular weight has higher acid-releasing ability.

### SUMMARY OF INVENTION

### Technical Problem

When PGA particles with a small number-average molecular weight Mn are added, the acid release rate is too high, so the shape of the medical film cannot be maintained for a predetermined period of time. Based on new findings by the present inventors, PGA is decomposed and its molecular weight decreases during production of the medical film. Therefore, when a film is produced using low-molecular-weight PGA particles, the molecular weight of the PGA particles in the film further decreases, so the acid release rate increases, and the shape retention effect of the medical film is significantly reduced. Therefore, in order to sufficiently enhance the shape retention effect of the medical film, a larger amount of PGA particles having a large number-average molecular weight Mn should be blended. However, when a large amount of high-molecular-weight PGA particles is blended, the dispersion of the PGA particles in the medical film tends to become non-uniform.

Here, immediately after the medical film is wetted with water, the bioabsorbable polymer compound swells and its shape changes, whereas the PGA particles do not undergo much shape change immediately after being wetted with water. Due to the difference in the degree of the shape change, interfacial defects that inevitably exist between the bioabsorbable polymer compound and the PGA particles increase immediately after wetting with water, causing a problem that the vulnerability of the film increases and the tensile strength decreases. As a result, stress generated when an organ moves or when the film is attached or peeled off causes fracture or tearing starting from these interfacial defects. Consequently, once a medical film has been attached to an organ, it is very difficult to peel off the film and reattach it again. The above problem has also occurred when the PGA particles had a large average particle size, or even when the average particle size was small, in cases where coarse aggregates resulting from non-uniform dispersion of the particles were present.

In addition, an adhesion barrier film is used by grasping the film with tweezers or the like during open abdominal surgery and attaching the film to an organ. Alternatively, when an adhesion barrier film is used by inserting the film during laparoscopic surgery, the film is rolled into a tubular shape, grasped with forceps, introduced into the body, then deployed (returned to its original shape), and attached to an organ. Therefore, the adhesion barrier film is required to not easily droop when grasped with tweezers or the like until the attachment or when grasped with forceps, and furthermore, is required to be easily rollable and to be easily deployable in the body. However, when a large amount of high-molecular-weight PGA particles is blended, the medical film tends to become rigid, which makes it difficult to roll up the film and/or difficult to deploy the film when the film is inserted into a living body or when the film is reattached.

An object of the present invention is to provide a medical film containing particles containing polyglycolic acid and a method for producing the medical film, the medical film being capable of retaining its shape in a living body for a long time, easy to reattach to an organ, and flexible and easy to deploy.

### Solution to Problem

One aspect of the present invention for solving the above problem relates to a medical film of [1] to [5] below.

[1]A medical film containing: a bioabsorbable polymer compound; and polyglycolic acid dispersoids that are particles containing a polymer having a constituent unit derived from glycolic acid or aggregates of the particles, in which
a number-average molecular weight of the polymer of the polyglycolic acid dispersoids is 7,500 or more and 100,000 or less,
an average particle size of the particles is 1 μm or more and 60 μm or less,
a content of the polyglycolic acid dispersoids per 100 parts by weight of the bioabsorbable polymer compound is 2 parts by weight or more and 40 parts by weight or less, and
a coefficient of variation CV of the number of particles contained in rectangular regions is 0.6 or less, where the coefficient of variation CV is determined from the number of particles measured in each of 16 rectangular regions with a size of 5 mm×4 mm set so as not to overlap with each other on a surface of the medical film.

[2] The medical film according to [1], in which in the 16 rectangular regions, an average value of the number of aggregates with a diameter that is 5 or more times an average diameter of the polyglycolic acid dispersoids contained in the rectangular regions is 3 or less.

[3] The medical film according to [1] or [2], in which the medical film has loop stiffness corrected by a thickness of the medical film of 100 (N/10 mm)/mm³ or more and 1100 (N/10 mm)/mm³ or less.

[4] The medical film according to any of [1] to [3], in which the medical film has a tensile strength of 11.0 MPa or more and 100.0 MPa or less.

[5] The medical film according to any of [1] to [4], being an adhesion barrier material.

In addition, one aspect of the present invention for solving the above problem relates to a method for producing a medical film of [6] below.

[6]A method for producing a medical film, the method including:
(a) mixing a solution or dispersion containing a bioabsorbable polymer compound with particles containing a polymer derived from polyglycolic acid to form a composite of the bioabsorbable polymer compound and the particles, and
(b) forming the composite of the bioabsorbable polymer compound and the particles into a film, in which
a viscosity of the solution or dispersion containing the bioabsorbable polymer compound in step (a) is 2,000 mPa·s or more, the viscosity being measured at 25° C. and 10 rpm,
a number-average molecular weight of the polymer in step (a) is 15,000 or more and 400,000 or less, and
an average particle size of the particles in step (a) is 1 μm or more and 60 μm or less.

### Advantageous Effects of Invention

According to the present invention, there are provided a medical film containing particles containing polyglycolic acid and a method for producing the medical film, the medical film being capable of retaining its shape in a living body for a long time, easy to reattach to an organ, and flexible and easy to deploy.

### BRIEF DESCRIPTION OF DRAWINGS

FIG. 1A is a photograph showing an external appearance of a film 4 after immersion in a phosphate buffer solution (PB) of pH 8.0 for 45 hours, and

FIG. 1B is a photograph showing an external appearance of a film 11 after immersion in a phosphate buffer solution (PB) of pH 8.0 for 45 hours.

### DESCRIPTION OF EMBODIMENTS

### Medical Film

One embodiment of the present invention relates to a medical film (hereinafter also referred to simply as the “medical film”) containing: a bioabsorbable polymer compound; and polyglycolic acid (PGA) dispersoids that are particles containing a polymer having a constituent unit derived from glycolic acid (hereinafter also referred to simply as the “PGA particles”) or aggregates of the particles.

### Bioabsorbable Polymer Compound

The bioabsorbable polymer compound is preferably a polymer compound that swells when immersed in ultrapure water at 15 to 25° C. The bioabsorbable polymer compound can be produced by physically crosslinking or chemically crosslinking the same type or different types of polymer compounds and is typically a gel polymer. The bioabsorbable polymer compound is preferably composed of a polymer that has solubility after swelling in water.

The bioabsorbable polymer compound can be, for example, a crosslinked product of a polyanionic polymer. The polyanionic polymer may be a polymer having a repeating structure having an anionic group or may be a polymer produced by chemically modifying a polymer compound with an anionic substituent. The anionic group can be a carboxyl group, a hydroxy group, a sulfo group, or the like. The anionic group may partially form a salt with an alkali metal, such as sodium or potassium, or an alkaline earth metal, such as calcium or magnesium.

The polyanionic polymer may be a polysaccharide, a protein, or a synthetic polymer. Among them, a polysaccharide or a protein is preferred.

Examples of the saccharide as the polyanionic polymer include natural saccharides, such as pullulan, alginic acid, hyaluronic acid, chondroitin acid, dextran acid, and pectin, and saccharides having an anionic group introduced by modification, such as carboxymethyl amylose, carboxymethyl cellulose, carboxymethyl dextran, carboxymethyl starch, sulfated cellulose, and sulfated dextran. Among them, alginic acid, hyaluronic acid, carboxymethyl amylose, and carboxymethyl cellulose are preferred because they are easy to handle.

Examples of the protein as the polyanionic polymer include gelatin, collagen, albumin, fibrin, and chemically modified products thereof. The protein may be a polymer of amino acids having an anionic group (polyamino acid), such as polyglutamic acid and polyaspartic acid. Among them, an acid-treated gelatin having a carboxyl group formed by hydrolysis of an acid amide is preferred.

The crosslink of the polymer compound may be a physical crosslink or may be a chemical crosslink formed by reacting the polymer compounds directly or via another molecule. The crosslink of the polymer compound can be obtained by subjecting the polymer compound to a crosslinking treatment, such as ultraviolet light treatment, heat treatment, and treatment with an activating agent. Among them, treatment with an activating agent is preferred.

Examples of the activating agent include various carbodiimide compounds such as 1-ethyl-3-(3-dimethylaminopropyl)carbodiimide (EDC), N,N′-dicyclohexylcarbodiimide, N,N′-diisopropylcarbodiimide, and N-cyclohexyl-N′-(2-morpholinoethyl)carbodiimide metho-p-toluenesulfonic acid. For example, a carbodiimide compound nucleophilically attacks an anionic group (carboxyl group) of a polyanionic polymer to form an o-acylisourea intermediate (e.g., paragraph [0033] of WO 2021/201150). It is considered that this activated intermediate then forms an ester bond with another polyanionic polymer to cause crosslinking, thereby forming a three-dimensional network structure. Among them, EDC is preferred because it is easy to handle.

The treatment with the activating agent can be carried out by dissolving the polymer compound in water or an aqueous medium, adding the activating agent, and stirring the mixture. The state of water or the aqueous medium at this time may be adjusted according to the type of activating agent. For example, when EDC is used as the activating agent, it is preferable to appropriately add an acid, such as 0.01 M hydrochloric acid, to adjust the pH during the reaction to a low pH (e.g., pH 4.0 to 5.5) and to dissociate the anionic group of the polyanionic polymer.

The amount of the activating agent is preferably 0.5 eq. or more and 10 eq. or less, more preferably 1 eq. or more and 6 eq. or less, and even more preferably 2 eq. or more and 5 eq. or less per reactive functional group of the polymer compound (e.g., hyaluronic acid).

The crosslinking may be caused between a plurality of different types of polymers. The crosslinking at this time may be chemical crosslinking caused by reacting these polymers with each other or may be physical crosslinking caused by increasing intermolecular interaction with an activating agent. For example, from the viewpoint of safety, a crosslinked product of hyaluronic acid and carboxymethyl cellulose in combination can be used as the bioabsorbable polymer compound.

### PGA Dispersoids

The PGA dispersoids are particles containing a polymer containing PGA (hereinafter also referred to simply as the “PGA polymer”) or aggregates of the particles. In the production of the medical film, the bioabsorbable polymer compound and the PGA particles form a composite and then formed into a film (film formation). At this time, the PGA particles may aggregate to form aggregates. The medical film may contain only PGA particles, may contain only aggregates of the PGA particles, or may contain both of them.

The PGA dispersoids gradually and sustainably release an acid by hydrolysis when the medical film comes into contact with water by attaching the film to an organ or by other means. The sustainably released acid suppresses the disintegration of the bioabsorbable polymer compound and can extend the period during which the film can retain its structure in a living body.

The expression “containing PGA” means that the ratio of the constituent unit derived from glycolic acid to a total mass of the polymer is 40 mass % or more and 100 mass % or less.

In the PGA polymer, the component other than PGA is not particularly limited as long as the ratio of the constituent unit derived from glycolic acid is 40 mass % or more. For example, the polymer can be a homopolymer of glycolic acid or a copolymer of glycolic acid and a hydroxycarboxylic acid, such as lactic acid, hydroxypropionic acid, hydroxybutyric acid, hydroxyvaleric acid, hydroxycaproic acid, or hydroxybenzoic acid, or a lactone, such as caprolactone. In addition, the PGA dispersoids may be a mixture with the above-mentioned polymer and a water-soluble polymer with confirmed biosafety, such as polyethylene glycol, or may be a copolymer of glycolic acid and polyethylene glycol or the like.

The ratio of the PGA polymer contained in the PGA dispersoids is preferably 50 mass % or more and 100 mass % or less, more preferably 60 mass % or more and 100 mass % or less, even more preferably 70 mass % or more and 100 mass % or less, still more preferably 80 mass % or more and 100 mass % or less, yet more preferably 90 mass % or more and 100 mass % or less, and particularly preferably 100 mass % relative to a total mass of the PGA dispersoids.

The PGA polymer has a number-average molecular weight of preferably 7,500 or more and 100,000 or less, more preferably 8,000 or more and 80,000 or less, even more preferably 8,000 or more and 50,000 or less, still more preferably 8,000 or more and 30,000 or less, and particularly preferably 10,000 or more and 30,000 or less. Increasing the number-average molecular weight can suppress excessive release of the acid and extend the period during which the shape of the medical film can be retained. The number-average molecular weight of the PGA polymer may decrease during the production of the film. Therefore, the PGA polymer at the time of film production has a number-average molecular weight of preferably 15,000 or more and 400,000 or less, more preferably 30,000 or more and 300,000 or less, and even more preferably 50,000 or more and 200,000 or less.

The number-average molecular weight of the PGA polymer can be determined by measuring the number-average molecular weight of the PGA polymer in the PGA particles separated from the medical film in accordance with ISO 16014-1: 2012. Specifically, after the film is cut into 30 mm squares, the film is immersed in a 50-mL vial to which 4 mL of concentrated hydrochloric acid (35-37% (mass percent concentration) available from Kanto Chemical Co., Inc.) has been added, and sonicated for 30 minutes. The solution after the sonication is diluted by adding ultrapure water and filtered through a 0.45-μm membrane filter, and then the residue on the filter is vacuum-dried at 25° C. The dried residue is transferred to a 10-mL volumetric flask, 0.5 mL of super dehydrated dimethyl sulfoxide is added, and the mixture is heated in an oil bath at 160° C. for 5 minutes. After the volumetric flask is allowed to cool to room temperature, a solution of 5 mM CF₃COONa dissolved in hexafluoropropanol (HFIP) is added to the flask to a total volume of 10 mL, and insoluble matter is removed from the resulting mixture by filtration through a filter to prepare a sample solution. The sample solution is measured by gel permeation chromatography (GPC) (apparatus: Shodex GPC-104 available from Resonac Holdings Corporation; detector: RI; column: HFWIP-606M×2; flow rate: 0.40 mL/min; temperature: 40° C.) using poly(methyl methacrylate) (PMMA) as a standard material. From a chromatogram obtained by the measurement, the number-average molecular weight Mn is calculated by molecular weight calculation using a calibration curve.

The average particle size of the PGA particles is 1 μm or more and 60 μm or less. The PGA particles with a smaller average particle size have a larger surface area and a higher degradation rate of the PGA particles, and have a larger area of contact with the bioabsorbable polymer compound, and thus can retain the shape of the medical film after attachment to an organ for a longer period. In addition, as the average particle size of the PGA particles is smaller, it is possible to suppress the softening of the film (decrease in thickness-corrected loop stiffness), which is caused by interfacial defects inevitably existing between the bioabsorbable polymer compound and the PGA particles, and the decrease in the tensile strength of the film when wetted with water. On the other hand, from the viewpoint of suppressing excessive aggregation of the PGA particles and a decrease in dispersion uniformity due to the aggregation, the average particle size of the PGA particles is preferably large to some extent. Therefore, the average particle size of the PGA particles is preferably 5 μm or more, and more preferably 8 μm or more. On the other hand, from the viewpoint of suppressing fracture or tearing due to an increase in interfacial defects between the PGA particles and the bioabsorbable polymer compound when wetted with water, or making the medical film flexible to facilitate reattachment or deployment, the average particle size of the PGA particles is preferably 50 μm or less, more preferably 40 μm or less, and even more preferably 30 μm or less. The average particle size of the non-aggregating PGA particles does not change before and after the production of the film.

The average particle size of the PGA particles can be measured by observing 200 or more PGA particles that are not aggregated using a digital microscope (available from Keyence Corporation: VHX7000) at a magnification of 100 times to capture digital images. The captured digital image is read into image analysis software, and the lengths of the perimeters of the PGA particles are measured with an image analyzer. The arithmetic mean (average value) and the standard deviation of the lengths of the perimeters of the PGA particles are calculated, measured values with a difference from the average value of three or more times the standard deviation are excluded to determine the average perimeter length, and the equivalent diameter of the particles obtained by dividing the average perimeter length by the ratio (3.1416) of the circumference of a circle to its diameter is defined as the average particle size of the PGA particles.

The image processing is performed by reading the digital image into MIPAR (available from LightStone Corp.) and setting the conditions of the image analysis software as follows. “Smart Cluster” is set to “Fill Type: Class”, “Classes: 4 to 10”, “Edge Type: Dark to Bright”, “Edge Clean: 0 to 5”, and “Speed: 2”. “Basic Threshold” is set to “Value: 80 to 150”.

“Reject Features” is set to “Measurement: Area”, “Target: Objects”, “Edges: Include”, “Units: μm²”, “Threshold Value: 100”, and “Type: Reject Features </=”.

Increasing the mixing ratio of the PGA dispersoids to the bioabsorbable polymer compound can extend the shape retention period of the medical film in a living body, even when using PGA that has a large number-average molecular weight Mn and a relatively slow acid release rate. On the other hand, adjusting the mixing ratio to an appropriate range can suppress excessive aggregation of the PGA particles, increase the dispersion uniformity, and suppress resulting shortening of the shape retention period of the medical film. The mass ratio of the PGA dispersoids to 100 parts by mass of the bioabsorbable polymer compound is 2 parts by mass or more and 40 parts by mass or less, preferably 8 parts by mass or more and 30 parts by mass or less, more preferably 10 parts by mass or more and 30 parts by mass or less, even more preferably 13 parts by mass or more and 30 parts by mass or less, and particularly preferably 20 parts by mass or more and 30 parts by mass or less.

The mass ratio of the PGA dispersoids contained in the medical film can be determined by removing the bioabsorbable polymer compound, measuring the mass of the remaining particles as the mass of the PGA dispersoids and measuring the mass of the removed bioabsorbable polymer compound, and calculating the mass of the PGA dispersoids relative to the mass of the bioabsorbable polymer compound. For the bioabsorbable polymer compound that can be dissolved and removed, such as hyaluronic acid and its crosslinked product, the material can be dissolved and removed using an appropriate acid compound. Then, a value obtained by subtracting the mass of water contained in the film and the mass of the residue, such as the PGA dispersoids remaining after the removal, from the mass of the medical film before the removal can be defined as the mass of the bioabsorbable polymer compound.

In the medical film according to the present embodiment, excessive aggregation of the PGA particles is suppressed, and this results in uniform dispersion of the PGA particles. This makes it possible to uniformly retain the shape throughout the entire film. Furthermore, since coarse aggregates can be reduced, it is possible to suppress tearing of the film, softening of the film (decrease in thickness-corrected loop stiffness), and decrease in strength, which are caused by interfacial defects inevitably existing between the bioabsorbable polymer compound and the PGA particles.

In the present embodiment, a coefficient of variation is used as an index representing the dispersibility of the PGA particles in the medical film. The coefficient of variation is not affected by the average particle size of the PGA particles and thus can evaluate the dispersibility of particles with different average particle sizes.

Specifically, the medical film has a coefficient of variation CV of the number of particles contained in rectangular regions of 0.6 or less, where the coefficient of variation CV is determined from the number of PGA particles measured in each of 16 rectangular regions with a size of 5 mm×4 mm set so as not to overlap with each other on a surface of the medical film. The coefficient of variation CV is an index indicating the degree of uniform dispersion of the PGA particles in the medical film. A smaller coefficient of variation CV indicates that the PGA particles are more uniformly dispersed in the medical film. The coefficient of variation CV is preferably 0.5 or less, more preferably 0.4 or less, even more preferably 0.3 or less, and particularly preferably 0.2 or less.

The coefficient of variation CV can be calculated by the following method.

On the surface of the medical film, 16 rectangular regions selected by the following method are set and observed at a magnification of 100 times using a digital microscope (available from Keyence Corporation: VHX7000) to capture digital images. The captured images are read into image analysis software, and the number of PGA particles contained in each rectangular region is determined from the lengths of the perimeters of the PGA dispersoids. Specifically, the average number of particles and the standard deviation of number of particles of the PGA particles in the 16 rectangular regions are calculated by the following method, and the calculated standard deviation is divided by the average number of particles to determine the coefficient of variation CV of the number of particles contained in the rectangular regions. The image analysis can be performed under the same conditions as in the calculation of the average particle size of the PGA particles described above.

At this time, one side of the medical film is defined as the x-axis, the other side of the film in the direction perpendicular to the x-axis is defined as the y-axis, and the intersection of the x-axis and the y-axis is defined as a coordinate point x0y0. The medical film is divided into five equal parts in the x-axis direction, and coordinate points x0y0, x1y0, x2y0, . . . , x5y0 are set from x0y0 along the x-axis. Similarly, the medical film is divided into five equal parts in the y-axis direction, and coordinate points x0y0, x0y1, x0y2, . . . , x0y5 are set from x0y0 along the y-axis. 16 rectangular regions each having a size of 5 mm×4 mm and containing 16 points of x1y1, x2y1, x3y1, x4y1, x1y2, x2y2, x3y2, x4y2, x1y3, x2y3, x3y3, x4y3, x1y4, x2y4, x3y4, and x4y4 in this coordinate plane are set so as not to overlap with each other, and each rectangular region is defined as a rectangular region i (i is an integer of 1 to 16). In addition, also when the plan view of the medical film is not substantially rectangular, 16 rectangular regions are equally defined from the entire planar direction of the medical film in the plan view according to the same concept.

Then, the length of the perimeter of a PGA dispersoid j observed in the rectangular region i is defined as L₍ⱼ₎. The PGA dispersoid and PGA particle are assumed to be spherical, and the diameter of the dispersoid is assumed to be proportional to the length of the perimeter of the PGA dispersoid, and the diameter of the PGA particle is assumed to be proportional to the length of the perimeter of the PGA particle. Based on this assumption, the number n₍ⱼ₎ of particles constituting the PGA dispersoid j is determined by Equation 1 from a length L₀ of the perimeter of one PGA particle and the length L₍ⱼ₎ of the perimeter of the PGA dispersoid j. In the following calculation, the length L₀ of the perimeter of the PGA particle determined by tentatively setting the average particle size or the lengths of the perimeters of the PGA particles is used. For example, in the following calculation, the average particle size of the PGA particles can be assumed to be 10 μm.

$$\begin{matrix}[ Math . 1 ] & \\ {n}_{( j )} = \frac{{L}_{( j )}^{3}}{{L}_{0}^{3}} & Equation 1\end{matrix}$$

From the number n₍ⱼ₎ of the PGA particles contained in each of N PGA dispersoids observed in the rectangular region i, the number X₍ᵢ₎ of the PGA particles contained in the rectangular region i is determined by Equation 2.

$$\begin{matrix}[ Math . 2 ] & \\ {X}_{( i )} = {\sum }_{j = 1}^{N} {n}_{( j )} = {\sum }_{j = 1}^{N} \frac{{L}_{( j )}^{3}}{{L}_{0}^{3}} & Equation 2\end{matrix}$$

An average number X₍ₐᵥₑ₎ of particles, which is the average value of the numbers of particles contained in each of the 16 rectangular regions, is determined by Equation 3.

$$\begin{matrix}[ Math . 3 ] & \\ {X}_{( ave )} = \frac{1}{16} {\sum }_{i = 1}^{16} {X}_{( i )} = \frac{1}{16} {\sum }_{i = 1}^{16} ( {\sum }_{j = 1}^{N} \frac{{L}_{( j )}^{3}}{{L}_{0}^{3}} ) & Equation 3\end{matrix}$$

Then, a standard deviation S of the number of particles observed in each rectangular region is determined, and the standard deviation S is divided by the average number X₍ₐᵥₑ₎ of particles to determine the coefficient of variation CV of the number of particles contained in the rectangular regions by Equation 4.

$$\begin{matrix}[ Math . 4 ] & \\ CV = \frac{S}{{X}_{( ave )}} = \frac{\sqrt{\frac{1}{16} {\sum }_{i = 1}^{16} {( {X}_{( i )} - {X}_{( ave )} )}^{2}}}{{X}_{( ave )}} & Equation 4\end{matrix}$$

The dispersibility of the PGA particles can also be evaluated by the average diameter of the PGA dispersoids in the medical film or the average value of the number of aggregates (hereinafter also referred to simply as the “5A aggregates”) with a diameter that is 5 or more times the average diameter of the PGA dispersoids in the rectangular region set as described above. Specifically, in the medical film, the average diameter of the PGA dispersoids is preferably 10 μm or more and 60 μm or less, more preferably 25 μm or more and 50 μm or less, and even more preferably 30 μm or more and 45 μm or less. In addition, in the medical film, the average value of the number of the 5A aggregates in the rectangular regions is preferably 3 or less, more preferably 2 or less, even more preferably 1 or less, and particularly preferably 0.

The average diameter of the PGA dispersoids and the average value of the number of the 5A aggregates in the rectangular regions can be calculated by the following methods.

Each rectangular region is observed at a magnification of 100 times using a digital microscope to capture a digital image. The captured digital image is read into image analysis software, and the lengths of the perimeters of the PGA dispersoids contained in each rectangular region are determined by the same method as that for calculation of the coefficient of variation CV. On the assumption that the PGA dispersoids are spheres, a diameter D₍ⱼ₎ of the j-th dispersoid i of N dispersoids observed in the rectangular region i is determined by Equation 5 by dividing the length L₍ⱼ₎ of the perimeter of the PGA dispersoid determined by image processing by the ratio π (3.1416) of the circumference of a circle to its diameter.

$$\begin{matrix}[ Math . 5 ] & \\ {D}_{( j )} = \frac{{L}_{( j )}}{π} & Equation 5\end{matrix}$$

Then, an average diameter D₍ᵢ₎ of the dispersoids contained in the rectangular region i is determined by Equation 6.

$$\begin{matrix}[ Math . 6 ] & \\ {D}_{( i )} = \frac{1}{N} {\sum }_{j = 1}^{N} {D}_{( j )} = \frac{1}{N \times π} {\sum }_{j = 1}^{N} {L}_{( j )} & Equation 6\end{matrix}$$

Furthermore, an average value D₍ₐᵥₑ₎ of the average diameters D₍ᵢ₎ of the dispersoids in the 16 rectangular regions is determined by Equation 7. In the present embodiment, this D₍ₐᵥₑ₎ is defined as the average diameter of the PGA dispersoids.

$$\begin{matrix}[ Math . 7 ] & \\ {D}_{( ave )} = \frac{1}{16} {\sum }_{i = 1}^{16} {D}_{( i )} & Equation 7\end{matrix}$$

In the same manner, in each of the 16 rectangular regions, an average diameter D₍ᵢ₎ of the aggregates and the number A₍ᵢ₎ of the aggregates with a diameter of 5 or more times D₍ᵢ₎ are measured. An average value A₍ₐᵥₑ₎ of the number A₍ᵢ₎ of the aggregates with a diameter of 5 or more times D₍ᵢ₎ in the 16 rectangular regions is determined by Equation 8. In the present embodiment, this A₍ₐᵥₑ₎ is defined as the average value of the number of the 5A aggregates in the rectangular regions.

$$\begin{matrix}[ Math . 8 ] & \\ {A}_{( ave )} = \frac{1}{16} {\sum }_{i = 1}^{16} {A}_{( i )} & Equation 8\end{matrix}$$

### Additional Component

The medical film may contain a lubricant, an emulsifier, a colorant, an antioxidant, a weather-resistant agent, a heat stabilizer, a crystal nucleating agent, an ultraviolet absorber, a colorant, an antibacterial agent, and/or the like. The content of these components is preferably 0 mass % or more and 20 mass % or less, more preferably 0 mass % or more and 10 mass % or less, and even more preferably 0 mass % or more and 5 mass % or less relative to the total mass of the medical film.

### Characteristics of Adhesion Barrier FILM

The adhesion barrier film preferably has thickness-corrected loop stiffness (i.e., loop stiffness corrected by a thickness, rigidity) of 100 (N/10 mm)/mm³ or more and 1100 (N/10 mm)/mm³ or less, more preferably 250 (N/10 mm)/mm³ or more and 1100 (N/10 mm)/mm³ or less, even more preferably 350 (N/10 mm)/mm³ or more and 900 (N/10 mm)/mm³ or less, particularly preferably 450 (N/10 mm)/mm³ or more and 900 (N/10 mm)/mm³ or less, and most preferably 650 (N/10 mm)/mm³ or more and 800 (N/10 mm)/mm³ or less. Loop stiffness is the resistance to bending and is an indicator of so-called “koshi” (firmness). When the loop stiffness of an adhesion barrier film corrected by the thickness of the film is 100 (N/10 mm)/mm³ or more, the film having such loop stiffness is appropriately less likely to bend and less likely to droop compared to a film having the same thickness. Therefore, even when the adhesion barrier film is grasped with forceps or the like for insertion into a living body, the film does not droop and easily retains its shape. Furthermore, after the bent or rolled up adhesion barrier film is inserted into a living body, the film easily returns to its original shape and thus is excellent in deployability. When the thickness-corrected loop stiffness is 1100 (N/10 mm)/mm³ or less, flexibility of the film can be ensured, so that the film is easily bent or rolled up even with a small force. In order to improve handleability of the adhesion barrier film, a balance between the flexibility and the deployability is important.

Loop stiffness is measured by the following method. For a rectangular adhesion barrier film, the film is divided into 4 divided areas by drawing a perpendicular line from the center of each side toward the opposing side. A sample piece can be prepared by cutting the film into a 10 mm×40 mm rectangle so as to include the central portion of each divided area. Here, the central portion of each divided area refers to the region including the intersection of the diagonals of a rectangle defining the divided area. However, when it is difficult to cut out sample pieces by the above method, the cutting method can be appropriately adjusted according to the dimensions of the film. Both ends of the rectangular film (sample piece) in the longitudinal direction are aligned and adhered to each other with double-sided tape at a width of 5 mm from each end to prepare a measurement sample in a loop shape (teardrop shape) with a loop length of 30 mm. The measurement is performed at a temperature of 25° C. and relative humidity of approximately 60% RH, and the portion adhered with the double-sided tape (5 mm) is clamped and fixed in the chuck of a creep meter (RHEONERII RE2-33005B available from Yamaden Co., Ltd.). To compress the loop of the measurement sample, the upper indenter (φ30 mm) attached to the creep meter is moved in the loop direction at a speed of 0.5 mm/sec. A load (N) at the time of compression by 5 mm from a contact point is recorded and defined as a loop stiffness value (N/10 mm). The measurement is performed four times (n=4), and the average of the measured values obtained is defined as the loop stiffness value. In a thin rectangular cross section such as a cross section of an adhesion barrier film, the second moment of area I is proportional to the cube of the thickness t, and therefore loop stiffness is easily affected by the thickness. In order to eliminate the influence of the thickness, correction is performed by Equation 9 using the thickness t (mm), and a thickness-corrected loop stiffness value Sₙₒᵣₘ ((N/10 mm)/mm³) is calculated.

$${S}_{norm} = \frac{S}{{t}^{3}} \qquad Equation 9$$

The adhesion barrier film has a thickness of preferably 10 μm or more and 500 μm or less, more preferably 10 μm or more and 200 μm or less, even more preferably 20 μm or more and 150 μm or less, and still more preferably 30 μm or more and 150 μm or less. As the thickness increases, the strength of the adhesion barrier film increases, and the film is less likely to be decomposed in a living body, so that the adhesion barrier effect can be exhibited for a long period. As the thickness decreases, the adhesion barrier film is easily bent or rolled up, and further handleability while being held with forceps or the like is easy.

The thickness of the adhesion barrier film is measured by the following method. For a rectangular adhesion barrier film, the film is divided into 16 divided areas by drawing perpendicular lines from points that divide each side into four equal segments toward an opposing side. The central portion of each divided area is measured with a micrometer (PMU150-25MX available from Mitutoyo Corporation), and the average value is defined as the film thickness. Here, the central portion of each divided area refers to the region including the intersection of the diagonals of a rectangle defining the divided area. The thickness of the above adhesion barrier film is the thickness in a dry state. The adhesion barrier film is usually in a dry state, but since the film may absorb moisture from the atmosphere, the thickness of the adhesion barrier film is measured in an environment at room temperature (25° C.) and humidity of 50% or less after removing moisture from the film by vacuum drying at room temperature (25° C.) for about 1 hour.

The tensile strength of the adhesion barrier film in a wet state with water is preferably 11.0 MPa or more and 100.0 MPa or less, more preferably 11.0 MPa or more and 80.0 MPa or less, even more preferably 11.0 MPa or more and 50.0 MPa or less, particularly preferably 11.0 MPa or more and 30.0 MPa or less, and most preferably 15.0 MPa or more and 30.0 MPa or less. In the present embodiment, the adhesion barrier film has a small amount of large-diameter PGA particles and also has a small amount of coarse aggregates caused by aggregation of the PGA particles. As a result, the tensile strength in a wet state with water can be increased.

The tensile strength is measured by the following method. The tensile strength of a sample wetted with ultrapure water is measured. In the measurement room, the temperature is 25° C. and the relative humidity is 60% RH. The sample is fixed to chucks so that the distance between the chucks is 20 mm. Berklin (product No. E-2 available from AION Co., Ltd.) soaked with ultrapure water is brought into close contact with a central portion of 10 mm×10 mm of one surface of the sample, and the sample is allowed to stand for 10 seconds. After 10 seconds, the sample is pulled at 0.1 mm/sec to measure the tensile strength (MPa).

### Method for Producing Medical Film

The medical film can be produced by crosslinking a polymer compound, forming a composite of the polymer compound and the PGA particles, and forming the composite into a film (film formation).

The crosslinking of the polymer compound is performed by a method suitable for the polymer, such as ultraviolet treatment, heat treatment, and treatment with an activating agent. For example, treatment with EDC as an activating agent can be performed by adding EDC to a solution of the polymer dissolved in water or an aqueous medium with stirring. At this time, an acid, such as HCl, is preferably added to adjust the solution to acidic conditions (e.g., pH 4.0 to 5.5). After the treatment with the activating agent, an unnecessary substance is preferably removed by dialysis or the like.

The composite with the PGA particles is formed by adding the PGA particles to a solution or dispersion containing the bioabsorbable polymer compound and stirring and mixing.

The film is formed by drying the composite of the bioabsorbable polymer compound and the PGA particles in a frame. The drying may be performed by air drying at ordinary temperature, freeze drying, or removing a solvent by heating. In addition, heat pressing (heating and pressurization) may be performed. The drying may be performed by combining a plurality of methods.

The temperature during drying is preferably 10° C. or more and 60° C. or less, more preferably 20° C. or more and 50° C. or less, and even more preferably 20° C. or more and 45° C. or less. When the temperature during drying is 20° C. or more and 50° C. or less, loop stiffness can be increased. The humidity (relative humidity RH) during drying is 35% RH or more and 95% RH or less, preferably 45% RH or more and 95% RH or less, more preferably 55% RH or more and 95% RH or less, even more preferably 60% RH or more and 95% RH or less, particularly preferably 70% RH or more and 95% RH or less, and most preferably 80% RH or more and 95% RH or less. The loop stiffness can be increased as the humidity during drying is increased.

The dried film-shaped composite may be further subjected to heat press treatment. By the heat treatment, an adhesion barrier film having further reduced solubility in water can be obtained. As a result, the period during which the structure is retained in a living body can be extended while maintaining the flexibility of the film. From the viewpoint of external appearance and handling properties, the heat treatment temperature is preferably 110° C. to 145° C. The heat treatment time is preferably 1 minute or more and 400 minutes or less, more preferably 1 minute or more and 200 minutes or less, and even more preferably 1 minute or more and 120 minutes or less. In addition, without performing the heat treatment, it is also possible to adjust solubility in water, for example, to increase solubility in water, by changing the content of the PGA particles, the form such as thickness, and/or the added amount of the activating agent relative to the bioabsorbable polymer compound.

On the other hand, according to the findings of the present inventors, blending a larger amount of PGA particles made of a PGA polymer having a high molecular weight to enhance the shape retention effect would promote aggregation of the PGA particles and non-uniform dispersion of the PGA particles. Excessive aggregation of the PGA particles and the resulting non-uniform dispersion would make it difficult to exhibit the shape retention effect of the film by the PGA particles. In addition, use of PGA particles with a large particle size or an increase of coarse aggregates due to aggregation of the PGA particles would reduce the tensile strength of the medical film when the film is wet with water and make the film prone to tearing. Furthermore, use of PGA particles with a large particle size or an increase in coarse aggregates due to aggregation of the PGA particles may make the film too flexible. Thus, this would make it difficult to attach the medical film as an adhesion barrier material to an organ or to reattach the medical film to an organ after being peeled off from another organ to which the film is once attached. In addition, the medical film attached to an organ as an adhesion barrier material may attach to another organ besides the target organ when the organ to which the film is attached or a human body moves. Thus, to extend the shape retention time of the medical film during which the shape can be retained in a living body and to make the medical film less prone to tearing also when the medical film is wet with water, it is important to uniformly disperse the PGA particles and suppress excessive aggregation.

In addition, in the formation of the composite, the dispersibility of the PGA particles can be adjusted to suppress excessive aggregation by adjusting the viscosity of the solution or dispersion containing the bioabsorbable polymer compound, the average particle size of the PGA particles, and/or the amount of the PGA particles to be added. From the above viewpoint, the viscosity of the solution or dispersion containing the bioabsorbable polymer compound measured at 25° C. and 10 rpm is preferably 2,000 mPa·s or more and 60,000 mPa·s or less, more preferably 3,000 mPa·s or more and 60,000 mPa·s or less, and even more preferably 5,000 mPa·s or more and 50,000 mPa·s or less. Furthermore, from the viewpoint of increasing the uniformity of thickness in forming the film, the viscosity of the solution or dispersion containing the bioabsorbable polymer compound measured at 25° C. and 1 rpm is preferably 5,000 mPa·s or more, more preferably 8,000 mPa·s or more and 300,000 mPa·s or less, and even more preferably 15,000 mPa·s or more and 150,000 mPa·s or less.

The particle sizes of the PGA particles for forming the composite can be adjusted by a known pulverization method. The known pulverization method is not particularly limited, but it is preferable to perform pulverization at a temperature lower than the glass transition temperature of the PGA polymer while applying a high shear force. As a device for pulverization (impact pulverization) while applying high shear force, any pulverization device may be used as long as impact force and shear force have a large contribution among the pulverization actions of compressive force, frictional force, impact force, and shearforce. For example, a ball mill, hammer mill, jet mill, cryomill, pin mill, or the like can be used. In addition to impact by a rotor, a turbo mill or the like that performs pulverization using ultra-high-speed vortex flow or high-frequency pressure vibration can be effectively utilized. From the perspective that pulverization efficiency and control of particle size are easy, a jet mill is preferred. In addition, for the purpose of removing fine powder, an air classifier may be combined, a pulverizer may be combined with a sieving machine to efficiently perform pulverization while controlling particle size, or a plurality of pulverization devices may be combined.

Usually, to increase the dispersibility of particulate matter added to sol, the viscosity of the sol is reduced. However, reducing the concentration by adding a solvent to reduce the sol viscosity would lead to movement of the dispersed PGA particles in the sol with a low viscosity during drying after casting, and this would promote aggregation and/or deviation in distribution of the PGA particles. In addition, to produce a film with a certain thickness or more when the concentration is reduced, the casting amount needs to be increased. Increasing the casting amount would result in uneven distribution of the sol during drying after casting and be likely to cause thickness deviation. Furthermore, increasing the casting amount would promote further aggregation and/or deviation in distribution of the particles due to the movement of the particles during drying. In contrast, in the present embodiment, increasing the viscosity of the sol prevents excessive aggregation of the particles and also makes it less likely to cause the thickness deviation of the film.

Moreover, increasing the viscosity of the solution or dispersion containing the bioabsorbable polymer compound can make the solution or dispersion less likely to spill from a casting frame when the solution or dispersion is placed in a dryer after casting, facilitating the production of the medical film. On the other hand, reducing the viscosity to some extent can facilitate the dispersion of the PGA particles and liquid feeding during production. Thus, a medical film with high dispersibility of the PGA particles can be produced by combining a sol with a viscosity as described above and PGA particles having an average particle size of 1 μm or more and 60 μm or less.

Adjusting these conditions can increase the dispersibility of the PGA particles and can reduce the amount of coarse aggregates in which the PGA particles aggregate in the medical film. In addition, this can suppress the shortening of the period during which the structure can be retained in a living body, the decrease in strength in a living body, and the like due to the aggregation of the PGA particles.

The film thus produced can be used as a medical film.

### Applications

The medical film can be attached to an organ or the like, starts to change in shape (disintegrate) by degradation after a predetermined time has elapsed, and is finally absorbed into a living body. The period from the attachment to the occurrence of the shape change can be adjusted to a period of 3 days to 14 days. In addition, the period from the attachment to the time when the medical film is absorbed into a living body and becomes invisible can be adjusted to a period of 14 days to 28 days. These periods can be adjusted according to the type of bioabsorbable polymer compound and the type of biodegradable resin.

The characteristics described above enable the medical film to be used as a covering material for an affected area for covering a wound site caused by surgery to prevent adhesion (adhesion barrier material), for preventing the occurrence of an ulcer or a perforation, and for other purposes. The medical film can also be used as a substrate for sustained release of a drug or a substrate for tissue generation. Alternatively, the medical film can also be used as a cavity holding material for holding a cavity by being applied to a wall surface of a lumen in a living body.

When used for various applications, the medical film may be used alone or used as a laminate with another layer.

### OTHER EMBODIMENTS

The embodiments described above are exemplary embodiments of the present invention, and needless to say, the present invention can include embodiments other than the embodiments described above within the scope of the technical idea constituting the core of the present invention.

### Examples

The present invention will be described in detail below based on examples, but the present invention is not limited to these examples.

### 1. Production of Medical Film

### 1-1. Production of Bioabsorbable Polymer Compound

### 1-1-1. Production of HA/CMC Condensate-1

Hyaluronic acid (HA) (available from Kewpie Corporation, molecular weight 2,200,000 to 2,500,000) in an amount of 1,650 mg and 750 mg of carboxymethyl cellulose (CMC) (available from Tokyo Chemical Industry Co., Ltd., molecular weight 250,000, degree of etherification from 0.5 to 0.8) were weighed. HA and CMC were added to a beaker containing 300 mL of ultrapure water and stirred until completely dissolved. Then, 0.1 N HCl was added to adjust the pH of the content to 4.80, and an HA/CMC solution was prepared. 1-Ethyl-3-(3-dimethylaminopropyl)carbodiimide (EDC) (available from Dojindo Laboratories) in an amount of 3,180 mg was weighed and dissolved in 7.5 mL of ultrapure water, and an EDC solution was prepared. While the HA/CMC solution was stirred at a stirring speed of 600 rpm and an appropriate amount of 0.1 N HCl was added to prepare the pH to 4.70 to 5.10, the entire volume of the EDC solution was added. The mixture was stirred for about 120 minutes until the pH did not change, and a reaction solution was obtained. Finally, the reaction solution after stirring was placed in a dialysis membrane with a molecular weight cut-off of 12,000 to 14,000 and stirred in ultrapure water. The entire volume of the dialysate (ultrapure water) was exchanged three times during 36 hours from the start of dialysis, and an HA/CMC condensate-1 (a solution of a bioabsorbable polymer compound) was obtained.

The concentration of the HA/CMC condensate-1 ((mass of HA and mass of CMC used as materials)/(mass of HA, CMC, and ultrapure water used as materials)) was 0.77 wt. %, the solution viscosity measured at 25° C. and 1 rpm was about 30,000 mPa·s, and the viscosity measured at 10 rpm was about 8,000 mPa·s.

The HA/CMC condensate-1 was diluted with ultrapure water, and an HA/CMC condensate-2 (a solution of a bioabsorbable polymer compound) was obtained. The concentration of the HA/CMC condensate-2 ((mass of HA and mass of CMC used as materials)/(mass of HA, CMC, ultrapure water used as materials, and ultrapure water for dilution)) was 0.67 wt. %, the solution viscosity measured at 25° C. and 1 rpm was about 11,000 mPa·s, and the viscosity measured at 10 rpm was about 4,000 mPa·s.

The HA/CMC condensate-1 was diluted with ultrapure water, and an HA/CMC condensate-3 (a solution of a bioabsorbable polymer compound) was obtained. The concentration of the HA/CMC condensate-3 ((mass of HA and mass of CMC used as materials)/(mass of HA, CMC, ultrapure water used as materials, and ultrapure water for dilution)) was 0.41 wt. %, the solution viscosity measured at 25° C. and 1 rpm was about 1,500 mPa·s, and the viscosity measured at 10 rpm was about 950 mPa·s.

### 1-2. Production of Medical Film

Particles of biodegradable polyesters shown in Table 1 were prepared. The following particles were used: particles 1 to 5 and 8 to 11 that passed through a sieve with an opening of 38 μm, particles 6 that passed through a sieve with an opening of 75 μm but did not pass through the sieve with an opening of 38 μm, and particles 7 that did not pass through the sieve with an opening of 75 μm.

The average particle size of the biodegradable polyester particles before the production of the film was measured by the following method.

That is, 200 or more biodegradable polyester particles that were not aggregated were observed using a digital microscope (available from Keyence Corporation: VHX7000) at a magnification of 100 times to capture digital images. The captured digital image was read into image analysis software, and the lengths of the perimeters of the PGA particles were measured with an image analyzer. The arithmetic mean (average value) and the standard deviation of the lengths of the perimeters of the biodegradable polyester particles were calculated, measured values with a difference from the average value of three or more times the standard deviation were excluded to determine the average perimeter length, and the equivalent diameter of the particles obtained by dividing the average perimeter length by the ratio (3.1416) of the circumference of a circle to its diameter was defined as the average particle size of the biodegradable polyester particles.

The image processing was performed by reading the digital image into MIPAR (available from LightStone Corp.) and setting the conditions of the image analysis software as follows. “Smart Cluster” was set to “Fill Type: Class”, “Classes: 4 to 10”, “Edge Type: Dark to Bright”, “Edge Clean: 0 to 5”, and “Speed: 2”. “Basic Threshold” was set to “Value: 80 to 150”. “Reject Features” is set to “Measurement: Area”, “Target: Objects”, “Edges: Include”, “Units: μm²”, “Threshold Value: 100”, and “Type: Reject Features </=”.

TABLE 1

| | | Raw material | | | |
|---|---|---|---|---|---|
| | | composition | | Number- | Average |
| | | (mass %) | | average | particle |
| Particle | | Glycolic | Lactic | molecular | size |
| Composition | Resin name | acid | acid | weight | (μm) |
| Particle 1 | Polyglycolic | 100 | 0 | 5,000 | 2.56 |
| | acid (PGA) | | | | |
| | powder | | | | |
| Particle 2 | Polyglycolic | 100 | 0 | 11,000 | 4.62 |
| | acid (PGA) | | | | |
| | powder | | | | |
| Particle 3 | Polyglycolic | 100 | 0 | 30,000 | 6.86 |
| | acid (PGA) | | | | |
| | powder | | | | |
| Particle 4 | Polyglycolic | 100 | 0 | 164,000 | 9.85 |
| | acid (PGA) | | | | |
| | powder | | | | |
| Particle 5 | Polyglycolic | 100 | 0 | 80,000 | 21.9 |
| | acid (PGA) | | | | |
| | powder | | | | |
| Particle 6 | Polyglycolic | 100 | 0 | 80,000 | 40.1 |
| | acid (PGA) | | | | |
| | powder | | | | |
| Particle 7 | Polyglycolic | 100 | 0 | 80,000 | 86.2 |
| | acid (PGA) | | | | |
| | powder | | | | |
| Particle 8 | Polyglycolic | 100 | 0 | 35,000 | 23.9 |
| | acid (PGA) | | | | |
| | powder | | | | |
| Particle 9 | Polyglycolic | 100 | 0 | 150,000 | 23.5 |
| | acid (PGA) | | | | |
| | powder | | | | |
| Particle 10 | Polylactic acid | 0 | 100 | 10,000 to | 5.31 |
| | polymer (PLA) | | | 18,000 | |
| | powder | | | | |
| Particle 11 | Polylactic acid | 0 | 100 | 100,000 | 6.79 |
| | polymer (PLA) | | | | |
| | powder | | | | |

### 1-2-1. Production of Film 1

The HA/CMC condensate-1 in an amount of 29.8 g and 62.7 mg of biodegradable polyester particles (particles 3) were weighed and added to a beaker. The content of the beaker to which the particles 3 were added was sonicated for 1 minute with stirring with a spatula. A 60×60 mm frame (frame thickness of 20 mm) was prepared on a glass plate, and the HA/CMC condensate-1 to which the particles 3 were added was poured into the frame. At a temperature of 30° C., the glass plate was placed on a rotating sample table and dried for about 3 days while the glass plate was rotated. After drying, a film was peeled off from the glass plate and heat-treated using a heat press machine at 90° C. for 15 minutes and then at 120° C. for 10 minutes, and a film 1 was obtained.

### 1-2-2. Production of Film 2

A film 2 was obtained in the same manner as in the production of the film 1 except for changing the biodegradable polyester particles to particles 4 and adding 10.5 mg of the particles 4.

### 1-2-3. Production of Film 3

A film 3 was obtained in the same manner as in the production of the film 1 except for changing the biodegradable polyester particles to the particles 4 and adding 20.9 mg of the particles 4.

### 1-2-4. Production of Film 4

A film 4 was obtained in the same manner as in the production of the film 1 except for changing the biodegradable polyester particles to the particles 4 and adding 62.7 mg of the particles 4.

### 1-2-5. Production of Film 5

A film 5 was obtained in the same manner as in the production of the film 1 except for adding 59.6 g of the HA/CMC condensate-1 and adding 62.7 mg of the particles 4.

### 1-2-6. Production of Film 6

A film 6 was obtained in the same manner as in the production of the film 1 except for changing the HA/CMC condensate-1 to the HA/CMC condensate-2, adding 33.8 g of the HA/CMC condensate-2, and adding 10.5 mg of the particles 4.

### 1-2-7. Production of Film 7

A film 7 was obtained in the same manner as in the production of the film 1 except for changing the biodegradable polyester particles to particles 5.

### 1-2-8. Production of Film 8

A film 8 was obtained in the same manner as in the production of the film 1 except for changing the biodegradable polyester particles to particles 6.

### 1-2-9. Production of Film 9

A film 9 was obtained in the same manner as in the production of the film 1 except for changing the biodegradable polyester particles to particles 8.

### 1-2-10. Production of Film 10

A film 10 was obtained in the same manner as in the production of the film 1 except for changing the biodegradable polyester particles to particles 9.

### 1-2-11. Production of Film 11

A film 11 was obtained in the same manner as in the production of the film 1 except for changing the biodegradable polyester particles to particles 1.

### 1-2-12. Production of Film 12

A film 12 was obtained in the same manner as in the production of the film 1 except for adding 59.6 g of the HA/CMC condensate-1, changing the biodegradable polyester particles to the particles 1, and adding 67.7 mg of the particles 1.

### 1-2-13. Production of Film 13

A film 13 was obtained in the same manner as in the production of the film 1 except for changing the biodegradable polyester particles to particles 2.

### 1-2-14. Production of Film 14

A film 14 was obtained in the same manner as in the production of the film 1 except for changing the biodegradable polyester particles to particles 10.

### 1-2-15. Production of Film 15

A film 15 was obtained in the same manner as in the production of the film 1 except for changing the biodegradable polyester particles to particles 11.

### 1-2-16. Production of Film 16

A film 16 was obtained in the same manner as in the production of the film 1 except for changing the biodegradable polyester particles to particles 7.

### 1-2-17. Production of Film 17

A film 17 was obtained in the same manner as in the production of the film 1 except for changing the biodegradable polyester particles to the particles 4 and adding 2.1 mg of the particles 4.

### 1-2-18. Production of Film 18

A film 18 was obtained in the same manner as in the production of the film 1 except for changing the HA/CMC condensate-1 to the HA/CMC condensate-3, adding 55.8 g of the HA/CMC condensate-3, changing the biodegradable polyester particles to the particles 4, and adding 10.5 mg of the particles 4.

Table 2 shows the compositions of the HA/CMC condensates, the viscosities of the solutions, and the added amounts of the HA/CMC condensates used in the production of each sample; the compositions, particle sizes, and added amounts of the PGA particles used in the production of each sample; and the parts by weight of the particles added per 100 parts by weight of the HA/CMC.

TABLE 2

| | | | | | | | | Parts by |
|---|---|---|---|---|---|---|---|---|
| | | | | | | | | weight of |
| | | HA/CMC condensate | | | Particles | | | particles per |
| | | HA/CMC | Solution | Added | | Particle | Added | 100 parts by |
| | | concentration/ | viscosity/ | amount/ | Particle | size/ | amount/ | weight of |
| | Sample | wt. % | mPa · s | g | composition | μm | mg | HA/CMC |
| Example 1 | Film 1 | 0.77 | 8000 | 29.8 | Particle 3 | 6.86 | 62.7 | 27.3 |
| Example 2 | Film 2 | 0.77 | 8000 | 29.8 | Particle 4 | 9.85 | 10.5 | 4.6 |
| Example 3 | Film 3 | 0.77 | 8000 | 29.8 | Particle 4 | 9.85 | 20.9 | 9.1 |
| Example 4 | Film 4 | 0.77 | 8000 | 29.8 | Particle 4 | 9.85 | 62.7 | 27.3 |
| Example 5 | Film 5 | 0.77 | 8000 | 59.6 | Particle 4 | 9.85 | 62.7 | 13.7 |
| Example 6 | Film 6 | 0.67 | 4000 | 33.8 | Particle 4 | 9.85 | 10.5 | 4.6 |
| Example 7 | Film 7 | 0.77 | 8000 | 29.8 | Particle 5 | 21.9 | 62.7 | 27.3 |
| Example 8 | Film 8 | 0.77 | 8000 | 29.8 | Particle 6 | 40.1 | 62.7 | 27.3 |
| Example 9 | Film 9 | 0.77 | 8000 | 29.8 | Particle 8 | 23.9 | 62.7 | 27.3 |
| Example 10 | Film 10 | 0.77 | 8000 | 29.8 | Particle 9 | 23.5 | 62.7 | 27.3 |
| Comparative | Film 11 | 0.77 | 8000 | 29.8 | Particle 1 | 2.56 | 62.7 | 27.3 |
| Example 1 | | | | | | | | |
| Comparative | Film 12 | 0.77 | 8000 | 59.6 | Particle 1 | 2.56 | 67.7 | 14.8 |
| Example 2 | | | | | | | | |
| Comparative | Film 13 | 0.77 | 8000 | 29.8 | Particle 2 | 4.62 | 62.7 | 27.3 |
| Example 3 | | | | | | | | |
| Comparative | Film 14 | 0.77 | 8000 | 29.8 | Particle 10 | 5.31 | 62.7 | 27.3 |
| Example 4 | | | | | | | | |
| Comparative | Film 15 | 0.77 | 8000 | 29.8 | Particle 11 | 6.79 | 62.7 | 27.3 |
| Example 5 | | | | | | | | |
| Comparative | Film 16 | 0.77 | 8000 | 29.8 | Particle 7 | 86.2 | 62.7 | 27.3 |
| Example 6 | | | | | | | | |
| Comparative | Film 17 | 0.77 | 8000 | 29.8 | Particle 4 | 9.85 | 2.1 | 0.9 |
| Example 7 | | | | | | | | |
| Comparative | Film 18 | 0.41 | 950 | 55.8 | Particle 4 | 9.85 | 10.5 | 4.6 |
| Example 8 | | | | | | | | |

### 2. Evaluation

### 2-1. Number-Average Molecular Weight Mn of PGA in Film

The number-average molecular weight of the PGA polymer in the PGA particles separated from each film was determined by measurement in accordance with ISO 16014-1: 2012. Specifically, after the film is cut into 30 mm squares, the film is immersed in a 50-mL vial to which 4 mL of concentrated hydrochloric acid (35-37% (mass percent concentration) available from Kanto Chemical Co., Inc.) has been added, and sonicated for 30 minutes. The solution after the sonication was diluted by adding ultrapure water and filtered through a 0.45-μm membrane filter, and then the residue on the filter was vacuum-dried at 25° C. The dried residue was transferred to a 10-mL volumetric flask, 0.5 mL of super dehydrated dimethyl sulfoxide was added, and the mixture was heated in an oil bath at 160° C. for 5 minutes. After the volumetric flask was allowed to cool to room temperature, a solution of 5 mM CF₃COONa dissolved in hexafluoropropanol (HFIP) was added to the flask to a total volume of 10 mL, and insoluble matter was removed from the obtained liquid mixture by filtration through a filter to prepare a sample solution. The sample solution was measured by gel permeation chromatography (GPC) (apparatus: Shodex GPC-104 available from Resonac Holdings Corporation; detector: RI; column: HFWIP-606M×2; flow rate: 0.40 mL/min; temperature: 40° C.) using poly(methyl methacrylate) (PMMA) as a standard material. From a chromatogram obtained by the measurement, the number-average molecular weight Mn was calculated by molecular weight calculation using a calibration curve.

### 2-2. Calculation of Coefficient of Variation of Number of Particles

On each surface of films 1 to 18, 16 rectangular regions selected by the following method were set and observed at a magnification of 100 times using a digital microscope (available from Keyence Corporation: VHX7000), and digital images were captured. The captured images were read into image analysis software (available from Lightstone Corp.: MIPAR), and the number of the biodegradable polyester particles contained in each rectangular region was determined from the lengths of the perimeters of the particles or their aggregates (dispersoids). The average number of particles and the standard deviation of number of particles in the rectangular regions of the 16 rectangular regions were calculated by the following method, the standard deviation was divided by the average number of particles, and the coefficient of variation CV of the number of particles contained in the rectangular regions was determined.

In identifying the dispersoids and measuring the lengths of the perimeters, each condition of the image analysis software was set as follows. “Smart Cluster” was set to “Fill Type: Class”, “Classes: 4 to 10”, “Edge Type: Dark to Bright”, “Edge Clean: 0 to 5”, and “Speed: 2”. “Basic Threshold” was set to “Value: 80 to 150”.

At this time, one side of the medical film was defined as the x-axis, the other side of the film in the direction perpendicular to the x-axis was defined as the y-axis, and the intersection of the x-axis and the y-axis was expressed as a coordinate point x0y0. The film was divided into five equal parts in the x-axis direction, and coordinate points x0y0, x1y0, x2y0, . . . , x5y0 were set from x0y0 along the x-axis. Similarly, the film was divided into five equal parts in the y-axis direction, and coordinate points x0y0, x0y1, x0y2, . . . , x0y5 were set from x0y0 along the y-axis. Rectangular regions each having a size of 5 mm×4 mm and containing 16 points of x1y1, x2y1, x3y1, x4y1, x1y2, x2y2, x3y2, x4y2, x1y3, x2y3, x3y3, x4y3, x1y4, x2y4, x3y4, and x4y4 in this coordinate plane were set so as not to overlap with each other and were each defined as a rectangular region i (i is an integer of 1 to 16). The length of the perimeter of the dispersoid j observed in the rectangular region i was defined as L₍ⱼ₎. The dispersoid and particle were assumed to be spherical, and the diameter of the dispersoid was assumed to be proportional to the length of the perimeter of the dispersoid, and the diameter of the particle was assumed to be proportional to the length of the perimeter of the particle. Based on this assumption, the number n₍ⱼ₎ of particles constituting the dispersoid j was determined by Equation 1 from a length L₀ of the perimeter of one particle and the length L₍ⱼ₎ of the perimeter of the dispersoid j. The length L₀ of the perimeter of the particle was determined on the assumption that the average particle size of the particles was 10 μm.

$$\begin{matrix}[ Math . 9 ] & \\ {n}_{( j )} = \frac{{L}_{( j )}^{3}}{{L}_{0}^{3}} & Equation 1\end{matrix}$$

From the number n₍ⱼ₎ of particles contained in each of N dispersoids observed in the rectangular region i, the number X₍ᵢ₎ of particles contained in the rectangular region i was determined by Equation 2.

$$\begin{matrix}[ Math . 10 ] & \\ {X}_{( i )} = {\sum }_{j = 1}^{N} {n}_{( j )} = {\sum }_{j = 1}^{N} \frac{{L}_{( j )}^{3}}{{L}_{0}^{3}} & Equation 2\end{matrix}$$

An average number X₍ₐᵥₑ₎ of particles, which was the average value of the numbers of particles contained in each of the 16 rectangular regions, was determined by Equation 3.

$$\begin{matrix}[ Math . 11 ] & \\ {X}_{( ave )} = \frac{1}{16} {\sum }_{i = 1}^{16} {X}_{( i )} = \frac{1}{16} {\sum }_{i = 1}^{16} ( {\sum }_{j = 1}^{N} \frac{{L}_{( j )}^{3}}{{L}_{0}^{3}} ) & Equation 3\end{matrix}$$

A standard deviation S of the number of particles observed in each rectangular region was determined, the standard deviation S was divided by the average number X₍ₐᵥₑ₎ of particles, and the coefficient of variation CV of the number of particles contained in the rectangular regions was determined by Equation 4.

$$\begin{matrix}[ Math . 12 ] & \\ CV = \frac{S}{{X}_{( ave )}} = \frac{\sqrt{\frac{1}{16} {\sum }_{i = 1}^{16} {( {X}_{( i )} - {X}_{( ave )} )}^{2}}}{{X}_{( ave )}} & Equation 4\end{matrix}$$

### 2-3. Average Diameter of Aggregates

Each rectangular region was observed using a digital microscope (available from Keyence Corporation: VHX7000) at a magnification of 100 times, and a digital image was obtained. The resulting digital image was read into image analysis software, and the lengths of the perimeters of the dispersoids contained in each rectangular region were determined by the same method as that for calculation of the coefficient of variation CV On the assumption that the dispersoids were spheres, the diameter D₍ⱼ₎ of the j-th dispersoid i of N dispersoids observed in the rectangular region i was determined by Equation 5 by dividing the length L₍ⱼ₎ of the perimeter of the dispersoid determined by image processing by the ratio π (3.1416) of the circumference of a circle to its diameter.

$$\begin{matrix}[ Math . 13 ] & \\ {D}_{( j )} = \frac{{L}_{( j )}}{π} & Equation 5\end{matrix}$$

Then, an average diameter D₍ᵢ₎ of the dispersoids contained in the rectangular region i was determined by Equation 6. Furthermore, an average value D₍ₐᵥₑ₎ of the average diameters D₍ᵢ₎ of the dispersoids in the 16 rectangular regions was determined by Equation 7.

$$\begin{matrix}[ Math . 14 ] & \\ {D}_{( i )} = \frac{1}{N} {\sum }_{j = 1}^{N} {D}_{( j )} = \frac{1}{N \times π} {\sum }_{j = 1}^{N} {L}_{( j )} & Equation 6\end{matrix}$$

$$\begin{matrix}[ Math . 15 ] & \\ {D}_{( ave )} = \frac{1}{16} {\sum }_{i = 1}^{16} {D}_{( i )} & Equation 7\end{matrix}$$

### 2-4. Average Number of 5A Aggregates in Rectangular Region

In each of the 16 rectangular regions, an average diameter D₍ᵢ₎ of the aggregates and the number A₍ᵢ₎ of the aggregates with a diameter of 5 times or more of D₍ᵢ₎ were measured. An average value A₍ₐᵥₑ₎ of the number A₍ᵢ₎ of the aggregates with a diameter of 5 times or more of D₍ᵢ₎ in the 16 rectangular regions was determined by Equation 8.

$$\begin{matrix}[ Math . 16 ] & \\ {A}_{( ave )} = \frac{1}{16} {\sum }_{i = 1}^{16} {A}_{( i )} & Equation 8\end{matrix}$$

### 2-5. Degradability

In 8 mL of a phosphate buffer solution (pH 8.0), a 100 mm² sample (dimension: 10 mm×10 mm) of each material of films 1 to 18 was immersed and stored at 37° C. The external appearance of the shape of the sample was observed after 24 h, 40 h, and 45 h elapsed from the immersion, and the shape was evaluated. A sample with a score of 2 points or more according to the rating below was determined to retain a film-like shape.

For the results of the external appearance observation, the following scores were set from the image of the external appearance of each sample, and the results were expressed by average values of the scores determined by a plurality (5) of experienced and skilled technicians with reference to the images.
4 Points: the external appearance is substantially the same as the planar shape of the sample before the immersion
3 Points: in the external appearance, the sample is entirely expanded from the planar shape before the immersion, but the presence of the sample can be visually confirmed
2 Points: in the external appearance, the sample is entirely expanded from the planar shape before the immersion and includes slightly disintegrated part, but the presence of the sample can be visually confirmed
1 Point: in the external appearance, the sample is entirely disintegrated or confirmed to be an indeterminate form with a droplet
0 Points: the sample is dissolved, and the external appearance cannot be confirmed

### 2-6. Strength

The films 1 to 18 were cut into strips with a long side of 40 mm and a short side of 10 mm, and these were used as samples. The tensile strengths of the samples wetted with ultrapure water were measured using a creep meter (RE2-33005B available from Yamaden Co., Ltd.). In the measurement room, the temperature was 25° C. and the relative humidity was 60% RH. The sample was fixed to chucks so that the distance between the chucks was 20 mm. Berklin (product No. E-2 available from AION Co., Ltd.) soaked with ultrapure water was brought into close contact with a central portion of 10 mm×10 mm of one surface of the sample, and the sample was allowed to stand for 10 seconds. After 10 seconds, the sample was pulled at 0.1 mm/sec to measure the tensile strength (MPa).

### 2-7. Thickness

The thickness of the adhesion barrier film was measured by the following method. For a rectangular adhesion barrier film, the film was divided into 16 divided areas by drawing perpendicular lines from points that divide each side into four equal segments toward an opposing side. The central portion of each divided area was measured with a micrometer (PMU150-25MX available from Mitutoyo Corporation), and the average value is defined as the film thickness. Here, the central portion of each divided area refers to the region including the intersection of the diagonals of a rectangle defining the divided area.

### 2-8. Loop Stiffness

Loop stiffness was measured by the following method. For a rectangular adhesion barrier film, the film is divided into 4 divided areas by drawing a perpendicular line from the center of each side toward the opposing side. A sample piece was prepared by cutting the film into a 10 mm×40 mm rectangle so as to include the central portion of each divided area. Both ends of the rectangular film (sample piece) in the longitudinal direction were aligned and adhered to each other with double-sided tape at a width of 5 mm from each end to prepare a measurement sample in a loop shape (teardrop shape) with a loop length of 30 mm. The measurement was performed at a temperature of 25° C. and a relative humidity of about 60% RH. A portion (5 mm) adhered with the double-sided tape was clamped and fixed in the chuck of a creep meter (RHEONER II RE2-33005B available from Yamaden Co., Ltd.). To compress the loop of the measurement sample, the upper indenter (φ30 mm) attached to the creep meter was moved in the loop direction at a speed of 0.5 mm/sec. A load (N) at the time of compression by 5 mm from a contact point was recorded and defined as a loop stiffness value (N/10 mm). The measurement was performed four times (n=4), and the average of the measured values obtained was defined as the loop stiffness value. In order to eliminate the influence of the thickness, correction was performed by Equation 9 using the thickness t (mm), and a thickness-corrected loop stiffness value Sₙₒᵣₘ ((N/10 mm)/mm³) was calculated.

$${S}_{norm} = \frac{S}{{t}^{3}} \qquad Equation 9$$

The results of Evaluations 2-1 to 2-8 of the films 1 to 18 are shown in Table 3.

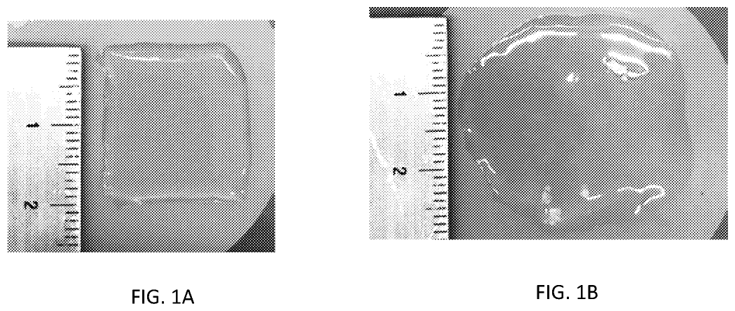

FIG. 1A is a photograph showing an external appearance of the film 4 after immersion for 45 hours, and FIG. 1B is a photograph showing an external appearance of the film 11 after immersion for 45 hours.

TABLE 3

| | | | | | | | | Tensile | | |
|---|---|---|---|---|---|---|---|---|---|---|
| | Number- | | | | | | | strength | | |
| | average | | | Average | | | | after | | |
| | molecular | Coefficient | | number of | | | | contact | | Loop |
| | weight | of | Average | 5A | | | | with water | | Stiffness |
| | Mn of | variation | diameter | aggregates | Degradability | | | for 10 | Thickness/ | (N/10 mm)/ |
| | PGA | CV | D₍ₐᵥₑ₎/μm | A₍ₐᵥₑ₎ | 24 h | 40 h | 45 h | sec/MPa | μm | mm³ |
| Example 1 | 8041 | 0.41 | 31.7 | 2 | 4 | 2 | 2 | 16.3 | 96.5 | 446.2 |
| Example 2 | 28213 | 0.14 | 34.4 | 1 | 4 | 2 | 2 | 19.9 | 62.8 | 753.8 |
| Example 3 | 29227 | 0.23 | 27.1 | 0 | 4 | 2 | 2 | 13.6 | 80.2 | 572.6 |
| Example 4 | 28825 | 0.39 | 32.6 | 0 | 4 | 3 | 2 | 22.5 | 78.0 | 545.8 |
| Example 5 | 27731 | 0.29 | 33.3 | 1 | 3 | 2 | 2 | 78.1 | 112.2 | 282.0 |
| Example 6 | 26866 | 0.47 | 37.0 | 3 | 4 | 2 | 2 | 14.8 | 55.0 | 1015.8 |
| Example 7 | 13527 | 0.40 | 32.7 | 1 | 4 | 2 | 2 | 15.0 | 60.2 | 720.8 |
| Example 8 | 17066 | 0.41 | 50.4 | 0 | 4 | 2 | 2 | 12.9 | 101.5 | 182.7 |
| Example 9 | 9382 | 0.40 | 32.7 | 1 | 4 | 2 | 2 | 14.4 | 81.7 | 563.6 |
| Example 10 | 24472 | 0.43 | 32.6 | 1 | 4 | 2 | 2 | 13.7 | 91.8 | 284.1 |
| Comparative | 6548 | 1.70 | 29.7 | 25 | 1 | 1 | 1 | 9.5 | 343.0 | 24.8 |
| Example 1 | | | | | | | | | | |
| Comparative | 5712 | 0.79 | 33.3 | 5 | 1 | 1 | 1 | 10.4 | 190.3 | 35.1 |
| Example 2 | | | | | | | | | | |
| Comparative | 7490 | 0.73 | 26.6 | 25 | 3 | 1 | 1 | 8.9 | 161.2 | 57.3 |
| Example 3 | | | | | | | | | | |
| Comparative | 9012 | 0.58 | 27.6 | 19 | 3 | 1 | 1 | 6.5 | 87.0 | 306.8 |
| Example 4 | | | | | | | | | | |
| Comparative | 49082 | 0.75 | 33.4 | 8 | 3 | 1 | 1 | 0.2 | 305.5 | 11.7 |
| Example 5 | | | | | | | | | | |
| Comparative | 26524 | 0.45 | 120.0 | 0 | 4 | 2 | 1 | 2.4 | 225.3 | 15.8 |
| Example 6 | | | | | | | | | | |
| Comparative | 27959 | 0.10 | 25.7 | 0 | 3 | 1 | 1 | 13.2 | 61.5 | 1109.2 |
| Example 7 | | | | | | | | | | |
| Comparative | 26984 | 0.85 | 43.2 | 16 | 3 | 1 | 1 | 2.5 | 67.8 | 628.0 |
| Example 8 | | | | | | | | | | |

*The number-average molecular weight of PGA in each of Comparative Examples 1 and 2 was higher than the number-average molecular weight of the PGA before the production of the film. It is considered that this is because PGA having a very low molecular weight was decomposed or eluted through the hydrochloric acid treatment and passed through the membrane filter, so that the number-average molecular weight of the residue on the filter apparently showed high values.

The results in Table 3 show that good dispersion of the PGA particles makes the film less soluble and helps retain the shape of the film in a living body. The results in Table 3 also show that the films with good dispersibility of the PGA particles have high tensile strength even in a wet state with water. A medical film with high tensile strength even in a wet state with water is less prone to tearing in a living body or the like and is easy to use in a living body.

The film 17 (Comparative Example 7) with the amount of the PGA particles of 0.9 parts by weight per 100 parts by weight of the bioabsorbable polymer compound was prone to disintegration despite the good dispersibility of the PGA particles. On the other hand, for the films 1 to 10 (Examples 1 to 10) with a small coefficient of variation CV of the number of particles contained in the rectangular regions despite the amount of the particles of 2 parts by weight or more, the disintegration of the film was delayed, and the disintegration of the film was suppressed.

Furthermore, the films 11 to 13 (Comparative Examples 1 to 3) with a large coefficient of variation CV of the number of particles contained in the rectangular regions despite containing the PGA particles were also prone to disintegration. Further, regarding the films 11 to 13, the thickness-corrected loop stiffness was low and the films were too flexible, and the tensile strength when wetted with water was low. In addition, the films 14 and 15 (Comparative Examples 4 and 5) containing polylactic acid particles had low dispersibility of the particles and were prone to disintegration. Furthermore, the film 16 (Comparative Example 6) having an average particle size of the PGA particles of 60 μm or more had low thickness-corrected loop stiffness and was too flexible, and the tensile strength when wetted with water was low.

### INDUSTRIAL APPLICABILITY

The medical film according to an embodiment of the present invention can retain its shape for a long period in a living body.

1. A medical film comprising:
a bioabsorbable polymer compound; and
polyglycolic acid dispersoids that are particles containing a polymer having a constituent unit derived from glycolic acid or aggregates of the particles, wherein
a number-average molecular weight of the polymer of the polyglycolic acid dispersoids is 7,500 or more and 100,000 or less,
an average particle size of the particles is 1 μm or more and 60 μm or less,
a content of the polyglycolic acid dispersoids per 100 parts by weight of the bioabsorbable polymer compound is 2 parts by weight or more and 40 parts by weight or less, and
a coefficient of variation CV of the number of particles contained in rectangular regions is 0.6 or less, where the coefficient of variation CV is determined from the number of particles measured in each of 16 rectangular regions with a size of 5 mm×4 mm set so as not to overlap with each other on a surface of the medical film.

2. The medical film according to claim 1, wherein in the 16 rectangular regions, an average value of the number of aggregates with a diameter that is 5 or more times an average diameter of the polyglycolic acid dispersoids contained in the rectangular regions is 3 or less.

3. The medical film according to claim 1, wherein the medical film has loop stiffness corrected by a thickness of the medical film of 100 (N/10 mm)/mm³ or more and 1100 (N/10 mm)/mm³ or less.

4. The medical film according to claim 1, wherein the medical film has a tensile strength of 11.0 MPa or more and 100.0 MPa or less.

5. The medical film according to claim 1, being an adhesion barrier material.

6. A method for producing a medical film, the method comprising:
(a) mixing a solution or dispersion containing a bioabsorbable polymer compound with particles containing a polymer derived from polyglycolic acid to form a composite of the bioabsorbable polymer compound and the particles, and
(b) forming the composite of the bioabsorbable polymer compound and the particles into a film, wherein
a viscosity of the solution or dispersion containing the bioabsorbable polymer compound in step (a) is 2,000 mPa·s or more, the viscosity being measured at 25° C. and 10 rpm,
a number-average molecular weight of the polymer in step (a) is 15,000 or more and 400,000 or less, and
an average particle size of the particles in step (a) is 1 μm or more and 60 μm or less.
